(19) United States
(12) Patent Application Publication
(10) Pub. No.: US 2009/0259195 A1
(43) Pub. Date: Oct. 15, 2009

(54) Prepacked automatic retractable safety syringe

(75) Inventor: Lee Lin Lee, Taipei (TW)

Correspondence Address: BACON & THOMAS, PLLC, 625 SLATERS LANE, FOURTH FLOOR, ALEXANDRIA, VA, 22314-1176, US

(73) Assignee: Bencha International Group. Inc., Tortola (VG)

(21) Appl. No.: 12/081,261

(22) Filed: Apr. 14, 2008

(51) Int. Cl. A61M 5/32

(52) U.S. Cl. 604/195

(57) ABSTRACT

This invention of a prepacked automatic retractable safety syringe comprises a needle hub with a needle tube and a rubber washer, a cap, a barrel, a spring, a vial, and a retractable plunger. To prevent or reduce injuries of needle stab and protect medical staffs, a forward action of the vial plus the needle hub compressed by the retractable plunger can drive a sliding movement between slides on the needle hub and snap hooks on the barrel via the spring's force to complete automatic retraction of the needle hub as well as the vial when an injection is finished.

### BACKGROUND OF THE INVENTION

1. Field of the Invention

The prepacked automatic retractable safety syringe is an invention of reducing injuries of needle stab and protecting a user's safety by loading liquor inside a vial in advance and depending on an action of a retractable plunger that can push the vial to compress a needle hub forward, then retracting the needle hub and the vial inside the barrel because of retraction of the retractable plunger and expansion of a spring when an injection is completed.

2. Description of the Prior Art

To control or clean various microbes within a human body and sift as well as examine diseases & symptoms, syringes, in general, are used as tools for injection and blood collection at a human body. Based on considerations in convenience in treatment, efficiency of management, correctness of pharmaceuticals, accuracy of doses, prevention of fake pharmaceuticals, and difference in products, utilization of prepacked injections has become an important tendency in development of the pharmaceutical industry. Despite a disposable barrel adopted for a conventional prepacked syringe with history of being used over 30 years, infected needle heads still are exposed. Thus far, there are 20 odd infectious diseases transmitted by blood like AIDS and B-type or C-type hepatitis with infected needle heads as channels that cause enormous economic and social costs for dealing with the sequential derivative problems after events of needle stab. In addition, patients having diabetes or users making injections punctually, after using syringes at other places such as offices, restaurants, or hotels, may discard used ones into common trash boxes or liter them everywhere, leading to exposure of infected needle heads that cause serious healthy problems in the society in case of injuries of needle stab to cleaners or children who catch needles.

Therefore, in addition to convenience in usage, the inventor, while actively researching safety syringes, deliberates healthy safety for users, cleaners, or even other people contacting infected needle head accidentally as a major objective in design that integrates a vial having prepacked injections in it with an automatic retractable syringe to keep a used needle hub and a vial retracted inside a barrel and to prevent exposure of an infected needle head causing injuries of needle stab to medical staffs, cleaners or any other people.

### SUMMARY OF THE INVENTION

This invention of prepacked automatic retractable safety syringe comprises a needle hub with a needle tube and a rubber washer on it, a cap, a barrel, a spring, a vial, and a retractable plunger. With an injection completed, a retraction from the retractable plunger pushing and compressing the needle hub makes the spring drive the needle hub plus the vial inside the barrel and materializes a safer design.

The major objective of this invention of a prepacked automatic retractable safety syringe is to furnish a vial with prepacked injections in it to make the vial contained inside an automatically retractable safety syringe. Because of plural U-type slides at the end of the needle hub as a design offering space for movement of plural snap hooks inside slides, and the spring around the needle hub together with a retractable plunger as a tool to push the vial compressing the needle hub forward, the spring is able to drive snap hooks moving inside slides of the needle hub and finally complete a retractable motion.

As another objective of this prepacked automatic retractable safety syringe to prevent injuries of needle stab and infection, the vial that contains prepacked injections and being held inside an automatically retractable safety syringe can be compressed by the forward moving retractable plunger to drive the spring retracting the needle hub, the vial, and the front plunger back to the barrel after completion of an injection.

Another objective of this prepacked automatic retractable safety syringe with the vial containing prepacked injections and being held inside an automatically retractable safety syringe is to form a complete retractable safety syringe that a rear plunger with a bulge on it can be engaged with the barrel when a front plunger is retracted inside the rear plunger.

With an illustration of diagrams, the detailed description and technical content related to this invention is displayed as follows:

### DETAILED DESCRIPTION OF THE PREFERRED EMBODIMENTS

Firstly, refer to FIG. 1, the 3-dimensional pictures of individual and combined components. This prepacked automatic retractable safety syringe having prepacked injections contained in a vial and held inside an automatically retractable safety syringe comprises: a needle hub 13 with a needle tube 130 and a rubber washer on it, a cap 10, a barrel 11, a vial 15 with injections preloaded in it, and a retractable plunger 16 that can be held inside the barrel 11. The barrel 11 is able to hold the needle hub 13 and guide the used needle hub 13 plus the vial 15 retracted toward a reversed direction against the injection by employing an annular spring 12.

Furthermore, the needle hub 13 containing a needle tube 131 with sharp heads at both ends and penetrating the center of the needle hub 13 has plural slides such as locating groove 131, ramp 132, rotation slide 133 and penetration slide 134 to form U-type slides for a retraction action. With snap hooks 110 at one end of the barrel 11 and the locating groove 131 forming a fastener, the snap hooks can be fixed inside the locating groove 131. When the spring 12 is further compressed, the snap hooks 110 can move along similar U-type slides formed by the locating groove 131, the ramp 132, the rotation slide 133, and the penetration slide 134 to make the needle hub 13 retracted to a reversed direction against the injection. At the other end of the needle hub 13, a disc-type retainer 135 with an annular concaved washer groove 1350 for cover of a rubber washer 14 has a function to contact inner circles inside an inner barrel 112 of the barrel 11 when the spring 12 is retracted.

Besides, the hollow barrel 11 has a fingergrip 111 at one end and plural inward snap hooks 110 with adequate tolerances for deformation at another end. When the syringe is unused, the snap hooks 110 securely hold the locating groove 131 of the needle hub 13; after the injection is finished, a retractable motion can be generated because a user's force along the direction of the needle hub 13 compresses the snap hooks 110 released from the groove between the needle hub 13 and the snap hooks 110.

Refer to FIGS. 2, 3, 4, 5 and 6 simultaneously, pictures for combination and movement of the vial and the retractable plunger in this preloaded automatic retractable safety syringe. The vial 15 for filling of liquor in advance is a hollow tube with a rubber stopper 150 and a hollow Π-type cover 151 installed at one end, and a gasket 152 with an adapter 1520 in it installed on the other end for the purpose of connecting a retractable plunger 16. The retractable plunger 16 is composed of a front plunger 160 and a rear plunger 161: the front plunger 160 is a hollow tube with one end open and a cylinder head 1600 formed in the center of one end for engagement of the cylinder head 1600 and the adapter 1520 of the gasket 152 inside the vial 15; the rear plunger 161 is a similarly hollow tube, which has an annular bulge 1610 and plural stop blocks 1612 around longitudinal walls on the tube for contact with the barrel 11.

At usage of this syringe, the cylinder head 1600 connecting to the front plunger 160 of the retractable plunger 16 is fixed inside the adapter 1520 of the gasket 152 within the vial 15, and the retractable plunger 16 for fixing the vial 15 is placed inside the inner barrel 112 of the barrel 11 for a forward movement action. When the lower point 1301 of the needle tube 130 penetrates the rubber stopper 150 of the vial 15, the retractable plunger 16 is able to propel injections inside the vial 15 into a human body through the needle tube 130. Then, with injection completed, a user's force to push the retractable plunger 16 toward the direction of the needle hub 13 will make the snap hooks 110 originally clamping the locating groove 131 of the needle hub 13 move at one end of the needle hub 13 along U-type slides formed by the ramp 132, the rotation slide 133, and the penetration slide 134, and lead to extension of the spring 12, retracting the needle hub 13 and the vial 15 toward the reversed direction against the injection.

Refer to FIGS. 7 & 8, pictures showing combination of the retractable plunger and the prepacked automatic retractable safety syringe. For the retractable plunger 16, the front plunger 160 with an annular raised rib 1601 around walls at one end, has a smaller diameter than a diameter of the rear plunger 161 with an annular concaved rib 1611 around walls of its inner edge. With this structure assembled, the retractable plunger 16 because of its plastic material with adequate intolerances for deformation has the raised rib 1601 securely contact the concaved rib 1611 and makes the front plunger 160 slide into the rear plunger 161 at compressing the retractable plunger 16;

For another case of embodiments of the retractable plunger 16′, because the front plunger 160′ with an annular concaved rib 1601′ around walls at one end, has a larger diameter than a diameter of the rear plunger 161′ with an annular raised rib 1611′ around walls of its inner edge, the concaved rib 1601′ of the front plunger 160′ is able to securely contact the raised rib 1611′ of the rear plunger 161′ when this structure is being assembled. In addition, when the retractable plunger 16′ is moved, the front plunger 160′ is able to slip into the outer rims of the rear plunger 161′.

Refer to FIGS. 9, 10 and 11, pictures showing movement of the retractable plunger of this prepacked automatic retractable safety syringe. When an injection is finished, a user's force compressing the retractable plunger 16 toward the needle hub 13 can make the snap hooks 110 originally engaging with the locating groove 131 of the needle hub 13 opened slightly, departing from the locating groove 131 and cause the raised rib 1601 of the front plunger 160 released from the concaved rib 1611 of the rear plunger 161 further owing to compression of a forward movement of the retractable plunger 16; on the other hand, adequate tension from the spring 12 retracting the needle hub 13, the vial 15, and the front plunger 160 toward the reversed direction against the injection makes the bulge 1610 around walls of the rear plunger 161 engage the concaved rib 1120 at one end of the inner barrel 112 inside the barrel 11, but a stop block 1612 located on the outer rim of the rear plunger 161 will be against one side of the fingergrip 111 to block the further movement of the rear plunger 161;

With the rear plunger 161 engaging the barrel 11, the function of the spring 12 to retract the needle hub 13, the vial 15 and the front plunger 160 inside the barrel 11 completes a cartridge-type safety syringe.

Refer to FIGS. 12, an operational view of another embodiment for the prepacked retractable safety syringe wherein the diameter of the front plunger 160′ of the retractable plunger 16 is greater than the diameter of the rear plunger 161′, and there is a hollow cylinder-type stopper 113′ formed at one side of the fingergrip 111′ of the syringe 11′ and a concaved rib 114′ formed around the inner circle inside the aperture. With the concaved rib 1601′ of the front plunger 160′ leaving the raised rib 1611 ′ of the rear plunger 161′ after an injection, the needle hub 13, the vial 15, and the front plunger 160′ can be retracted inside the barrel along a reversed direction against the injection due to extension of the spring 12. Additionally, the bottom plate 1613′ on one side of the rear plunger 161′ can be engaged with the concaved rib 114′ around the inner circle inside the stopper 113′ of the barrel 11′, creating an immobile contact when the needle hub 13, the vial 15, and the front plunger 160′ are retracted.

Refer to FIGS. 13 & 14, an operational view of another embodiment for the needle hub of the prepacked retractable safety syringe. Storage for the needle tube 130 penetrating the center of the needle hub 13 can be divided into two portions: the needle tube 130′ is contained inside one end of the needle hub 13; the needle tube 130″ is contained in the center of the retainer 135′ at another end of the needle hub 13′.

### BRIEF DESCRIPTION OF THE DRAWINGS

FIG. 1 is a three-dimensional exploded view for components of the prepacked automatic retractable safety syringe.

FIG. 2 is a plane view for components combination of the prepacked automatic retractable safety syringe.

FIG. 3 is a cross-sectional view of the retractable plunger engaging the vial of the prepacked automatic retractable safety syringe.

FIG. 4 is a cross-sectional view for combination of the vial and the needle hub of the prepacked automatic retractable safety syringe.

FIG. 5 is an operational view of the prepacked automatic retractable safety syringe.

FIG. 6 is an operational view for combination of the needle hub and snap hooks of the prepacked automatic retractable safety syringe.

FIG. 7 is a diagrammatical illustration of embodiment for combination of the retractable plunger of the prepacked automatic retractable safety syringe.

FIG. 8 is a diagrammatical illustration of another embodiment for the retractable plunger of the prepacked automatic retractable safety syringe.

FIG. 9 is an operational view for retraction of the retractable plunger of the prepacked automatic retractable safety syringe.

FIG. 10 is an operational view for retraction of the prepacked automatic retractable safety syringe.

FIG. 11 is a diagrammatical illustration with contraction completed for the prepacked automatic retractable safety syringe.

FIG. 12 is a diagrammatical illustration of another embodiment for retraction of the retractable plunger of the prepacked automatic retractable safety syringe.

FIG. 13 is a diagrammatical illustration of embodiment for the needle tube of the needle hub of the prepacked automatic retractable safety syringe.

FIG. 14 is a diagrammatical illustration of another embodiment for the needle tube of the needle hub of the prepacked automatic retractable safety syringe.

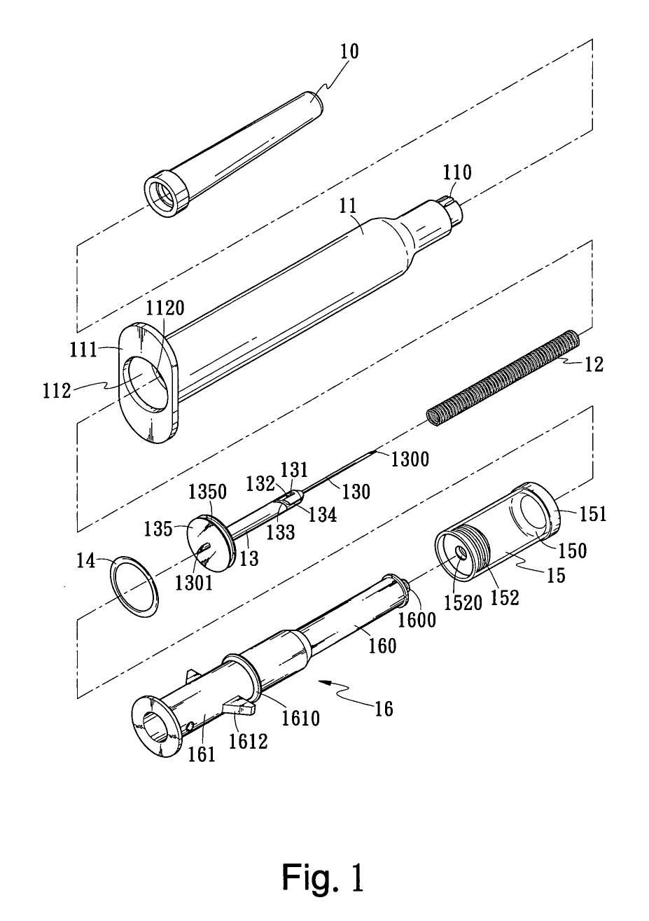

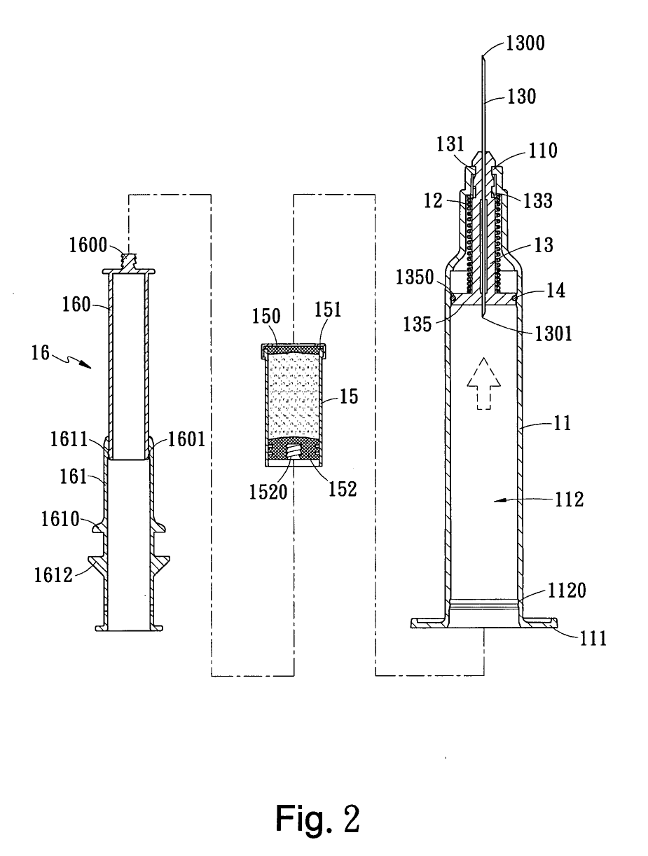

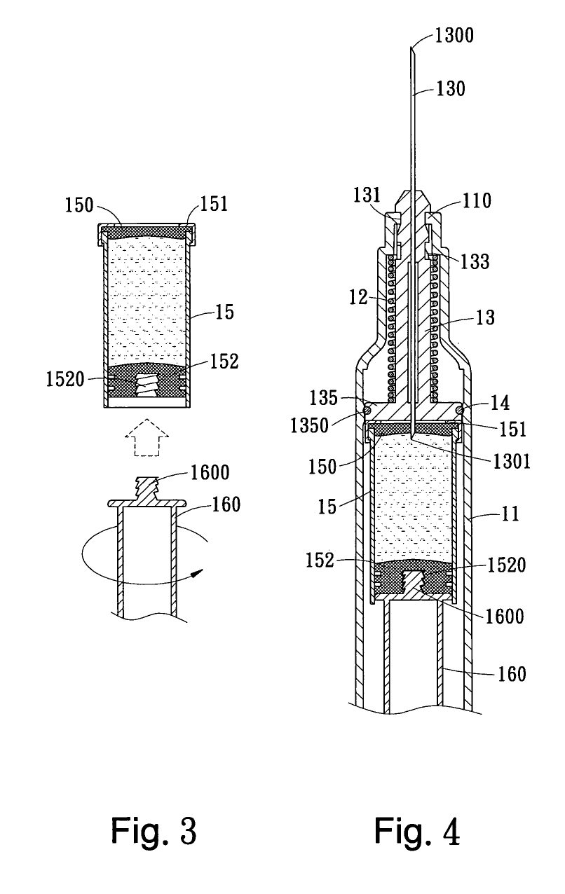

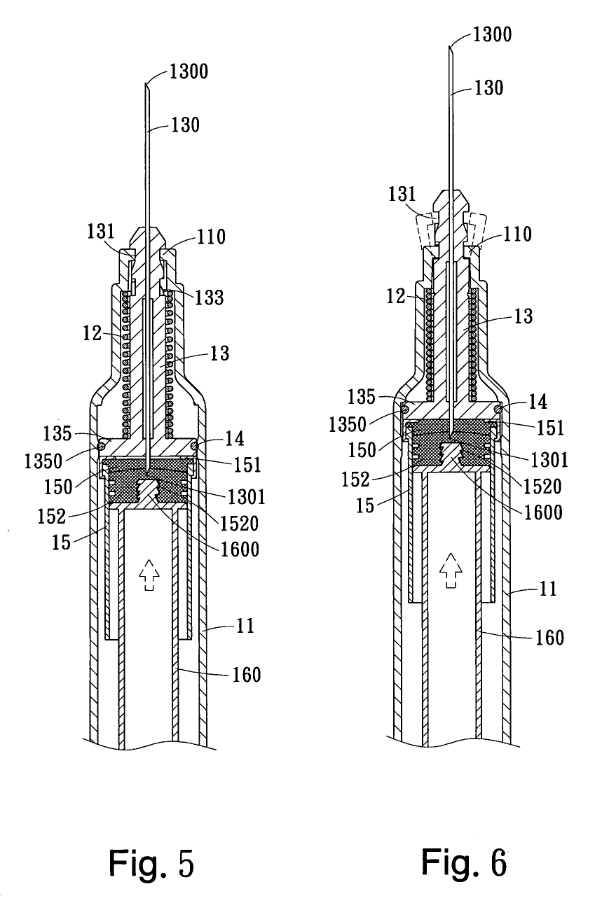

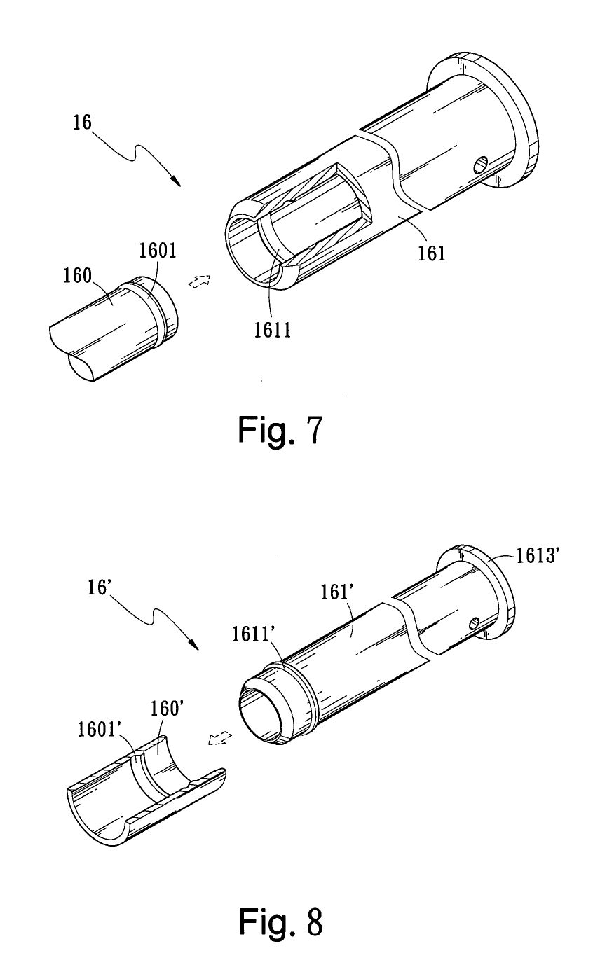

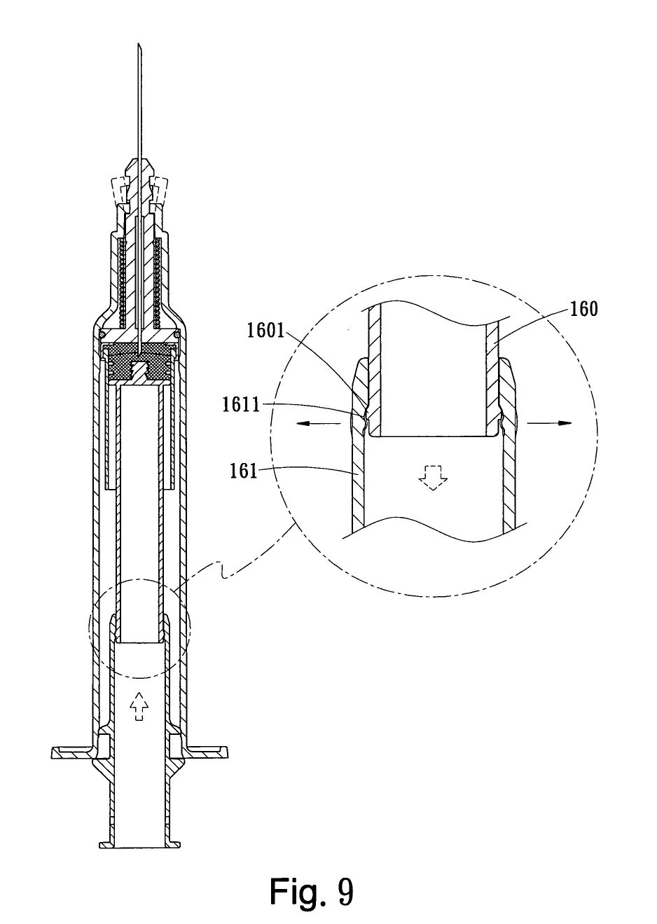

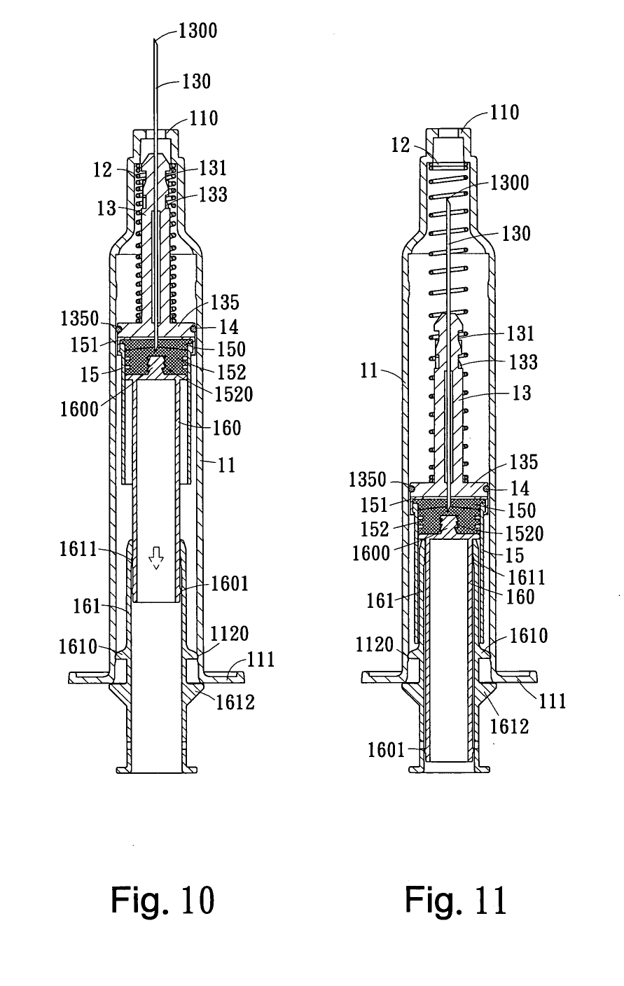

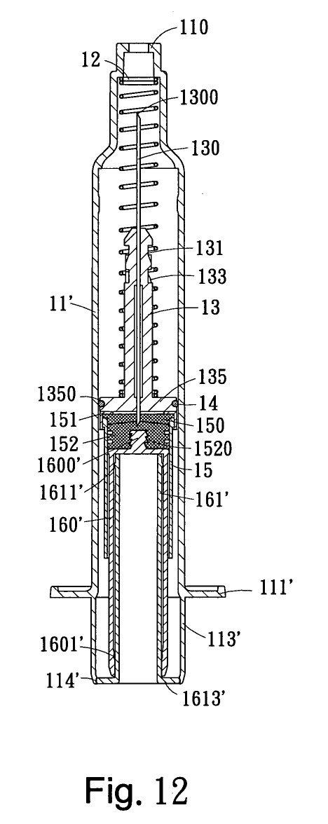

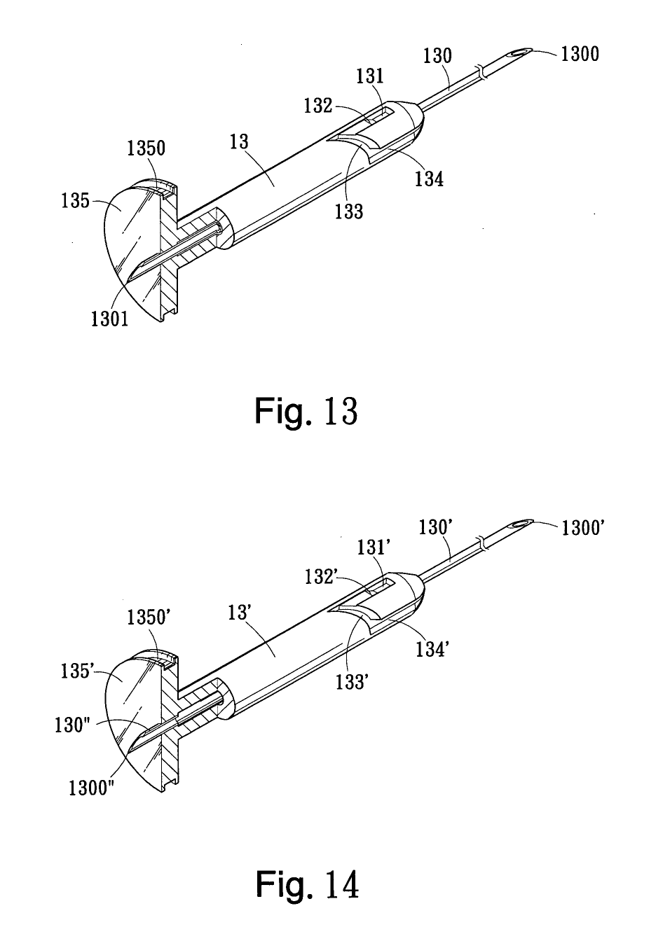

What is claimed is:

1. A prepacked automatic retractable safety syringe, which comprises: a barrel having plural snap hooks to clip a needle hub and guiding the needle hub plus a vial to retract inside the barrel with injection completed;
a needle hub with a twin-head sharp penetrating needle tube in the center to penetrate a vial for injection and plural U-type concaved slides at one end;
a spring around a needle hub to provide tension for retraction of the needle hub;
a vial preloaded liquor or powder medicine and having a rubber stopper, a cover, and a mobile gasket forming an adapter for engagement with a retractable plunger; and
a retractable plunger with two mutually engaged plungers that one of them has a greater diameter than another one and with a cylinder head at one end to be contained inside a barrel.

2. The prepacked automatic retractable safety syringe according to claim 1 wherein the needle tube on the needle hub can be contained at both ends of the needle hub.

3. The prepacked automatic retractable safety syringe according to claim 1 wherein the adapter within the gasket is designed as a spiral cone joint.

4. The prepacked automatic retractable safety syringe according to claim 1 wherein the retractable plunger has a front plunger with a smaller diameter around its outer circle than the diameter around the rear plunger's inner circle.

5. The prepacked automatic retractable safety syringe according to claim 1 wherein the retractable plunger has a front plunger with a larger diameter around its inner circle than the diameter around the rear plunger's outer circle.

6. The prepacked automatic retractable safety syringe according to claim 1 wherein the barrel has a concaved rib around its inner circle.

7. The prepacked automatic retractable safety syringe according to claim 1 wherein the barrel has a stopper at one end.

8. The prepacked automatic retractable safety syringe according to claim 7 wherein the stopper forms a concaved rib around an inner circle of an aperture side.

9. The prepacked automatic retractable safety syringe according to claim 1 wherein the cylinder head of the retractable plunger is designed as a spiral cone.

10. The prepacked automatic retractable safety syringe according to claim 1 wherein the vial can be a single cavity or a twin cavity.

11. A prepacked automatic retractable safety syringe, which comprises: a barrel having plural snap hooks to clip the needle hub and guiding the needle hub to retract inside the barrel with injection completed;
a needle hub with a twin-head sharp penetrating needle tube in the center and with a retainer covered by a rubber washer at one end, which has plural approximate U-type grooved slides;
a spring around the needle hub for retraction of a needle hub; and
a retractable plunger composed of two mutually engaged plungers that one of them has a larger diameter than another one and a rubber head formed at one plunger's end for becoming an identical body at a retraction action.

12. The prepacked automatic retractable safety syringe according to claim 11 wherein the needle tube in the needle hub can be contained on both sides of the needle hub.

13. The prepacked automatic retractable safety syringe according to claim 11 wherein the retractable plunger has a front plunger with a smaller diameter around its outer circle than the diameter around the rear plunger's inner circle.

14. The prepacked automatic retractable safety syringe according to claim 11 wherein the retractable plunger has a front plunger with a larger diameter around its inner circle than the diameter around the rear plunger's outer circle.

15. The prepacked automatic retractable safety syringe according to claim 11 wherein the barrel has a concaved rib inside its inner circle.

16. The prepacked automatic retractable safety syringe according to claim 11 wherein the barrel has a stopper at one end.

17. The prepacked automatic retractable safety syringe according to claim 16 wherein the stopper forms a concaved rib around an inner circle of an aperture side.
